(19) United States
(12) Patent Application Publication
(10) Pub. No.: US 2011/0184284 A1
(43) Pub. Date: Jul. 28, 2011

(54) NON-INVASIVE DEVICES AND METHODS TO DIAGNOSE PAIN GENERATORS

(75) Inventor: William F. McKay, Memphis, TN (US)

(73) Assignee: WARSAW ORTHOPEDIC, INC., Warsaw, IN (US)

(21) Appl. No.: 12/695,906

(22) Filed: Jan. 28, 2010

(51) Int. Cl. A61B 8/00

(52) U.S. Cl. 600/437

(57) ABSTRACT

Devices and methods are provided that diagnose one or more pain generators at or near the spine in a patient suffering from pain. In one embodiment, a device is provided that includes: an external transducer probe having a proximal end, a distal end and a body therebetween wherein the probe is configured to be placed in contact with the skin of a patient near a pain generator or suspected pain generator; an energy source that supplies the probe with energy; and a conduit for engaging the energy source and the proximal end of the probe. The energy is transferred from the energy source to the distal end of the probe through the skin of the patient and has an intensity to deliver and produce a pain signal when at least the distal end of the probe is near the pain generator or suspected pain generator.

### BACKGROUND

Pain can adversely affect patients in many different ways. It can keep the patient from being active, sleeping well, enjoying family and friends, and from eating. Pain can make the patient feel afraid or depressed and prevent full participation in general rehabilitation programs and may even slow recovery.

Pain serves the important biological function of signaling the presence of damage or disease within the body and is often accompanied by inflammation (redness, swelling, and/or burning). There are two categories of pain: acute pain and neuropathic pain. Acute pain refers to pain experienced when tissue is being damaged or is damaged. Acute pain serves at least two physiologically advantageous purposes. First, it warns of dangerous environmental stimuli (such as hot or sharp objects) by triggering reflexive responses that end contact with the dangerous stimuli. Second, if reflexive responses do not avoid dangerous environmental stimuli effectively, or tissue injury or infection otherwise results, acute pain facilitates recuperative behaviors. For example, acute pain associated with an injury or infection encourages an organism to protect the compromised area from further insult or use while the injury or infection heals. Once the dangerous environmental stimulus is removed, or the injury or infection has resolved, acute pain, having served its physiological purpose, ends. As contrasted to acute pain, in general, neuropathic pain serves no beneficial purpose. Neuropathic pain results when pain associated with an injury or infection continues in an area once the injury or infection has resolved.

There are many painful diseases or conditions that require proper pain and/or inflammation control including but not limited to a spinal disc herniation (i.e., sciatica), lower back pain, lower extremity pain, upper extremity pain, cancer, tissue pain and pain associated with injury or repair of cervical, thoracic, and/or lumbar vertebrae or intervertebral discs, facet joints, tendons, ligaments, muscles, spondilothesis, stenosis, discogenic back pain, or the like.

Many of the above conditions require surgical treatment to try to alleviate or control the pain. For example, an intervertebral disc herniation is a painful condition that may occur in any of the 33 discs of the spine but herniations in the lumbar and the cervical spine are most common. Disc herniations in the cervical spine may cause radiating pain and muscle dysfunction in the arm, which is generally referred to as cervical rhizopathy. Disc herniations in the lumbar spine may induce radiating pain and muscle dysfunction in the leg which is generally referred to as sciatica. Treatments for intervertebral disc herniations include open or mini-open surgery, using very small opening incisions or percutaneously, utilizing specially designed instruments and radiographic techniques to target the pain generator or area that is involved in the painful condition.

Unfortunately, particularly with pain generators in the spine, the cause for the pain may be difficult to diagnose, as there are numerous structures containing nociceptors and often the pain radiates throughout the body. To complicate matters for the practitioner, the vertebrae of the spine look very similar and are often no more than an inch tall with only a small separation between their bony structures. Sometimes, particularly when the spine is injured or abnormal, it may be difficult to locate the injured or abnormal vertebrae involved in causing the pain. Often times the practitioner will take additional steps such as taking several X-rays, MRIs and CAT scans, and counting the number of vertebrae to ensure the right vertebra is being treated. To diagnose a pain generator from a spinal disc, for example, the practitioner may perform an invasive discography, where a needle is inserted into a disc puncturing the annulus of the disc which may increase the incidence of disc degeneration.

In spite of these additional steps, sometimes the wrong vertebra is indeed treated which subjects the patient to additional surgeries. Other times, the test to diagnose the pain generator itself may increase the risk of disc degeneration. The patient will often feel afraid or depressed and this may prevent full participation in general rehabilitation programs and may even slow recovery. Thus, there is a need to improve the diagnosis of painful spinal diseases. There is also a need to reduce surgical errors from practitioners operating on the wrong tissue site of the spine.

### SUMMARY

Methods and devices are provided to properly diagnose the presence, location and intensity of one or more pain generators at or near the spine without invasively penetrating any disc tissue thereby helping to avoid any accelerated disc degeneration. The methods and devices provided herein also dictate the location of the surgery by properly locating the pain generators thereby reducing the risk that the practitioner operates on the wrong area of a patient. The methods and devices provided herein also enable a practitioner to produce a pain map that locates the pain generators so that the practitioner can rule out referred pain that is where the pain sensation is localized to an area completely unrelated to the site of injury.

The pain generator may be due to conditions involving pain and/or inflammation. The pain and/or inflammation may for example be due to chronic conditions including, a spinal disc herniation (e.g., sciatica), lower back pain, discogenic back pain, lower extremity pain, upper extremity pain, tissue pain and pain associated with injury or repair of cervical, thoracic, and/or lumbar vertebrae or intervertebral discs and facet joints, spondilothesis, stenosis or the like.

In one embodiment, a device for diagnosing a pain generator or a suspected pain generator at or near the spine in a patient suffering from pain is provided. The device comprises an external transducer probe having a proximal end, a distal end and a body therebetween; an energy source that supplies the probe with energy; and a conduit for engaging the energy source and the proximal end of the probe. The probe is configured to be placed in contact with the skin of the patient near the pain generator or suspected pain generator. The energy is transferred from the energy source to the distal end of the probe and has an intensity to deliver and produce a pain signal through the skin of the patient to the target tissue site at or near the pain generator or suspected pain generator when at least the distal end of the probe is pointed in the direction of the pain generator or suspected pain generator. The probe can further comprise a tip attached to its distal end to be placed in contact with the skin of the patient and the energy can be transferred to the tip from the energy source. The tip can be removable. The probe can further comprise a sensor to monitor the position of the energy source relative to the spine in the patient. If the probe comprises a sensor, the device can further comprise a stereotactic system having an overhead camera for monitoring the sensor. In some embodiments, the probe is re-positioned on the skin of a patient in the direction of the suspected pain generator until it is positioned on the skin of the patient at a position pointing in the direction of the pain generator so that the location of the pain generator is diagnosed. In some embodiments, the probe is re-positioned on the skin of a patient in the general direction of a plurality of suspected pain generators before it is re-positioned on the skin of a patient at a position in the specific direction of a plurality of pain generators so that locations of the pain generator can be mapped. The distal end of the probe, when contacting the skin of the patient, can vibrate a tissue having the pain generator or the suspected pain generator at or near the spine to generate a pain signal.

In another embodiment, a method for diagnosing a location of a pain generator or a suspected pain generator at or near the spine in a patient suffering from pain is provided. The method comprises: positioning an external transducer probe on the skin of a patient in the general direction of the pain generator or suspected pain generator wherein the probe has a proximal end, a distal end and a body therebetween and the probe is supplied with energy from an energy source via a conduit that connects the energy source to the proximal end of the probe; and delivering sufficient energy from the distal end of the probe through the skin of the patient to a tissue at or near the pain generator or suspected pain generator to produce a pain signal so as to diagnose the location of the pain generator or suspected pain generator. The probe can further comprise a tip attached to its distal end to be placed in contact with the skin of the patient and the energy can be transferred to the tip from the energy source. The tip can be removable. The probe can further comprise a sensor to monitor the position of the energy source relative to the spine in the patient. If the probe comprises a sensor, the device can further comprise a stereotactic system having an overhead camera for monitoring the sensor. In some embodiments, the probe can be re-positioned on the skin of the patient in a general direction of a suspected pain generator a plurality of times before it is positioned on the skin of a patient at a position in the specific direction of the pain generator so that the location of the pain generator is diagnosed. In some embodiments, the probe can be re-positioned at a plurality of suspected pain generators before it is re-positioned on the skin of the patient at a position in the specific direction of a plurality of pain generators so that the location of the pain generators can be mapped.

In yet another embodiment, a device for diagnosing a pain generator or a suspected pain generator at or near the spine in a patient suffering from pain is provided. The device comprises: (a) a first external transducer probe having a proximal end, a distal end and a body therebetween, the first probe configured to be placed in contact with the skin of the patient at a first location in the general direction of the pain generator or suspected pain generator; (b) a second external transducer probe having a proximal end, a distal end and a body therebetween, the second probe configured to be placed in contact with the skin of the patient at a second location in the general direction of the pain generator or suspected pain generator; (c) an energy source that supplies the first probe and the second probe with energy; (d) a first conduit for engaging the energy source and the proximal end of the first probe; and (e) a second conduit for engaging the energy source and the proximal end of the second probe. The energy is transferred from the energy source to the distal end of the first probe and the distal end of the second probe. The energy from both the first probe and the second probe is of an intensity for each probe to deliver a signal through the skin of the patient to or toward the pain generator or suspected pain generator when the distal end of each probe is pointing at the specific direction of the pain generator or the suspected pain generator and the signals produced by each probe intersect at the location of the pain generator or the suspected pain generator. The first probe can further comprise a removable tip attached to its distal end to be placed in contact with the skin of the patient and the second probe can further comprise a removable tip attached to its distal end to be placed in contact with the skin of the patient wherein the energy is transferred to the tip of the first probe from the energy source and the tip of the second probe from the energy source. Each probe can comprise a sensor to monitor the position of the energy source relative to the spine in the patient. The device can further comprise a stereotactic system having an overhead camera for monitoring the sensors.

The energy in the various embodiments can be ultrasound energy. The ultrasound energy can have a frequency in the approximate range of 15 kHz-20 MHz, 20 kHz-100 kHz, 25 kHz-50 kHz, 30 kHz-50 kHz, 1 MHz-10 MHz, 5 MHz-10 MHz or 500 kHz-1 MHz.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

In part, other aspects, features, benefits and advantages of the embodiments will be apparent with regard to the following description, appended claims and accompanying drawings where:

FIG. 1 illustrates a side plan view of an embodiment of a device comprising an external transducer probe engaged with an energy source via a conduit.

FIG. 2 illustrates a side plan view of an embodiment of an external transducer probe having a proximal end, a distal end and a body therebetween.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding the numerical ranges and parameters set forth herein, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The headings below are not meant to limit the disclosure in any way; embodiments under any one heading may be used in conjunction with embodiments under any other heading.

### Definitions

It is noted that, as used in this specification and the appended claims, the singular forms “a,” “an,” and “the,” include plural referents unless expressly and unequivocally limited to one referent. Thus, for example, reference to “a pain regenerator” includes one, two, three or more pain generators.

The term “diagnostic” or “diagnosing” means identifying the presence, absence, and/or location of one or more pain generators or suspected pain generators associated with the pain.

The term “at or near ” is intended to include a region extending up to and including from 0 cm to 5 cm from the target tissue site at or near the spine (e.g., nerve, muscle, ligament, bone, vertebra, etc.), as well as interior regions within the target tissue site.

The term “spine” includes neuronal, bony, vascular and soft tissue components. This includes the vertebral bodies and their associated joints (facets, costovertebral joints, or disc interfaces), the intervertebral discs, the intrinsic musculature, the spinal cord, spinal nerves, sympathetic nerves or ganglia associated with the axial skeleton, vertebral or disc innervations, and/or associated blood vessels.

The term “axial” refers to the head, neck and/or back of a patient.

The term “disc” may be one or more discs within a spinal column, including cervical, thoracic or lumbar discs.

The term “disc region” is intended to include a region extending about 5 cm from the surface of a disc, the surface of the disc, as well as interior regions within the disc.

The term “degeneration” refers to anatomical signs of degeneration, which can include changes in the height of the disc, the level of hydration of the disc, ruptured or contained herniation, annular bulging, and the presence of tearing or osteophytes. A reduction in the height of the disc may be one of the most common, early and easily detectable changes present in a degenerating disc. Another sign of degeneration is normally the loss of the T2 weighted signal on an MRI scan; this is indicative of a loss of hydration of the nuclear tissue. The degeneration can be a contained disc that occupies the space determined by the size of the endplates or a herniated disc. Herniation could be of a contained nature called, for example, bulging of the disc or a herniated disc can also be ruptured with release of discal elements, such as the nucleus pulposus, outside the disc. Signs of degeneration such as inflammation, tissue density, changes in pH, increased innervation and vascularization can also be found adjacent to the disc.

The term “radiculopathy” refers to radicular leg or arm pain derived from abnormalities of the disc or elements adjacent to the disc that can affect a spinal root or other neuronal elements within the spinal column.

The term “degeneration” refers to a traumatic or progressive abnormality linked to the development of an axial pain with and without radiculopathy condition. Suitable non-limiting examples of degeneration in the intervertebral disc or the area adjacent to the disc include bulging or protrusion, inflammation, pressure, changes in neuronal, vascular, immune or matrix elements, electrical activity, water content, tissue density, and changes in pH.

The term “pain” includes nociception and the sensation of pain, both of which can be assessed objectively and subjectively, using pain scores and other methods well-known in the art. A person of ordinary skill in the art will recognize that the threshold for pain may vary between different patients. Accordingly, the results of the diagnosing of the pain generator or suspected pain generator or the testing and/or monitoring of the pain generator or suspected pain generator may be correlated with the pain measurements according to techniques of pain assessment known in the art. Such correlation enables the practitioner to choose the course of treatment which better fits the needs of the patient. The techniques of pain assessment include, without limitation, VAS, Oswestri, and SF-36 Questionnaires, pain scores from 0 to 10, where 0 is none and 10 is most. These can be used to determine if a pain signal is produced. A “pain signal” includes the patient's perception of pain in response to the tissue having contact with an energy source such as ultrasonic energy. This pain signal can be induced by vibrating the tissue with energy such as ultrasonic energy.

Pain, as used herein, also includes allodynia (i.e., increased response to a normally non-noxious stimulus) and hyperalgesia (i.e., increased response to a normally noxious or unpleasant stimulus), which can in turn, be thermal or mechanical (tactile) in nature. In some embodiments, pain is characterized by thermal sensitivity, mechanical sensitivity and/or resting pain. In other embodiments, pain comprises mechanically-induced pain or resting pain. In still other embodiments, the pain comprises resting pain. The pain can be primary or secondary pain, as is well-known in the art. Exemplary types of pain that can be diagnosed and treated by the methods and devices of the present application include, without limitation, back pain in the lumbar regions (low back pain) or cervical region (neck pain), leg pain, sciatic pain, radicular pain (experienced in the lower back and leg from lumber pathology, or in the neck and arm from cervical pathology), or neuropathic pain of the arm, neck, back, lower back, leg, or related pain distributions resulting from disc or spine pathology. As used herein, “neuropathic pain” includes pain arising from injury to the nerve root, dorsal root ganglion or peripheral nerve.

The term “pain generator” refers to the source or cause involved in the painful condition. The term “suspected pain generator” includes the source or cause believed to be involved in the painful condition, yet no definitive diagnosis of the location of the pain generator has been made. Pain generators can occur from, for example, vertebral abnormalities, such as, compression fractures, pars defects, vertebral instability, soft tissue abnormalities in ligaments, tendons, annulus, muscles, cartilaginous structures, joints (e.g., facet joints, intervertebral discs, sacroiliac joints, etc.) or abnormalities resulting from tumors, infection or other infiltrative processes. Pain generators can result from nerve ingrowth into annulus tears and facet capsules, nerve root lesions (e.g., compressive lesions from adjacent discs, hypertrophic facet joints, facet joints cysts, faulty hardware positioning, bony foraminal encroachment, spondylolisthesis, spondylolysis, congenitally short pedicles, nerve sheath tumors, granulation tissue and/or arachnoiditis, etc.), spinal nerve compression (e.g., spinal stenosis), peripheral nerve lesions, femoral neuropathy, meralgia paresthetica, peroneal neuropathy, asymmetrical neuropathies, lower limb joint pathology, vascular pathology, degenerative disc and joint disease or the like. In some embodiments, the methods and devices provided can be used to locate the pain generator that causes persistent pain and/or radicular pain, which accompany underlying spinal abnormalities. The term “no pain generator” includes tissue that does not contain any pain generator.

The term “surgical procedure” includes a procedure in which one or more incisions are made into the body in order to repair damage or remove diseased tissue. In some embodiments, the surgical procedure can include implanting one or more drug depots or other implantable device.

The term “spinal surgery” includes a procedure in which one or more incisions are made and requires manipulation of spinal tissues, with or without removal and/or repair of spinal tissues. Examples of spinal surgery include, but are not limited to, repair of a herniated disc, adhesioloysis, radiofrequency neurotomy; intradiscal electrothermal therapy, fusion of vertebrae, full or partial discectomy, laminectomy, laminotomy, or laminoplasty, or the like.

The language “external transducer probe” includes a probe capable of propagating an energy emitted by an energy source through the skin of a patient and into a target tissue site to cause mechanical movement of the tissue.

The term “practitioner” means a person who is using the methods and/or devices of the current disclosure on the patient. This term includes, without limitation, doctors (e.g., surgeons, interventional specialists, physicians), nurses, nurse practitioners, other medical personnel, clinicians, veterinarians, or scientists.

The drug depot can comprise an analgesic or an anti-inflammatory agent. The phrase “anti-inflammatory agent” refers to an agent or compound that has anti-inflammatory effects. These agents may remedy pain by reducing inflammation. Anti-inflammatory agents also include those agents in a different classification with anti-inflammatory properties, such as, for example, amitriptyline, carbamazepine, gabapentin, pregabalin, clonidine, or other alpha adrenergic receptor agonist or a combination thereof.

The pain and/or inflammation may for example be due to chronic conditions including rheumatoid arthritis, osteoarthritis, a spinal disc herniation (e.g., sciatica), carpal/tarsal tunnel syndrome, lower back pain, discogenic back pain, lower extremity pain, upper extremity pain, cancer, tissue pain and pain associated with injury or repair of cervical, thoracic, and/or lumbar vertebrae or intervertebral discs, rotator cuff, articular joint, TMJ, tendons, ligaments, muscles, spondilothesis, stenosis, or joint pain or the like.

A “drug depot” is the composition in which at least one anti-inflammatory agent and/or at least one analgesic agent or the pharmaceutically acceptable salts of either or both are administered to the body. Thus, a drug depot may comprise a physical structure to facilitate implantation and retention in a desired site (e.g., a disc space, a spinal canal, a tissue of the patient, particularly at or near a site of surgery, pain, or site of inflammation, etc.). The drug depot also comprises the drug itself. The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API.” It will be understood that unless otherwise specified a “drug” formulation may include more than one therapeutic agent, wherein exemplary combinations of therapeutic agents include a combination of two or more drugs. The drug provides a concentration gradient of the therapeutic agent for delivery to the site. In various embodiments, the drug depot provides an optimal drug concentration gradient of the therapeutic agent at a distance of up to about 0.1 cm to about 5 cm from an implant site, and comprises at least one anti-inflammatory agent or its pharmaceutically acceptable salt and/or at least one analgesic agent or its pharmaceutically acceptable salt.

A “depot” includes but is not limited to capsules, microspheres, microparticles, microcapsules, microfibers particles, nanospheres, nanoparticles, coating, matrices, wafers, pills, pellets, emulsions, liposomes, micelles, gels, or other pharmaceutical delivery compositions or a combination thereof. In some embodiments, the depot can include a pump. Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof.

A “therapeutically effective amount” or “effective amount” is such that when administered, a drug results in alteration of the biological activity, such as, for example, inhibition of inflammation, reduction or alleviation of pain, improvement in the condition, etc. The dosage administered to a patient can unless otherwise specified or apparent from context be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments, the drug depot formulation is designed for immediate release. In other embodiments, the drug depot formulation is designed for sustained release. In other embodiments, the drug depot formulation comprises one or more immediate release surfaces and one or more sustain release surfaces.

The term “mammal” refers to organisms from the taxonomy class “mammalian,” including but not limited to humans, other primates such as chimpanzees, apes, orangutans and monkeys, rats, mice, cats, dogs, cows, horses, etc. In various embodiments, the mammal is a human patient.

Treating or treatment of a disease or condition refers to executing a protocol, which may include the use of a device herein and/or administering one or more drugs to a patient (human, normal or otherwise, or other mammal), in an effort to diagnose and alleviate signs or symptoms of the disease. Alleviation can occur prior to signs or symptoms of the disease or condition appearing, as well as after their appearance. Thus, “treating” or “treatment” includes “preventing” or “prevention” of disease or undesirable condition. In addition, “treating” or “treatment” does not require complete alleviation of signs or symptoms, does not require a cure, and specifically includes protocols that have only a marginal effect on the patient. “Reducing pain” includes a decrease in pain and does not require complete alleviation of pain signs or symptoms, and does not require a cure. In various embodiments, reducing pain includes even a marginal decrease in pain. By way of example, the administration of the effective dosages of at least one analgesic agent and/or at least one anti-inflammatory agent may be used to prevent, treat or relieve the symptoms of pain and/or inflammation.

“Localized” delivery includes delivery where energy is directed at the target tissue, for example, a nerve root of the nervous system or a region of the brain, or in close proximity (within about 5 cm, or preferably within about 2 cm, or within about 1 cm, or less for example) thereto.

### Device

In one embodiment, a device for diagnosing a pain generator or a suspected pain generator at or near the spine in a patient suffering from pain is provided. The device comprises an external transducer probe having a proximal end, a distal end and a body therebetween; an energy source that supplies the probe with energy; and a conduit for engaging the energy source and the proximal end of the probe. The probe is configured to be placed in contact with the skin of the patient near the pain generator or suspected pain generator. The energy is transferred from the energy source to the distal end of the probe and has an intensity to deliver and produce a pain signal through the skin of the patient at or near the pain generator or suspected pain generator when at least the distal end of the probe is pointing in the direction of the pain generator or suspected pain generator. The probe can further comprise a tip attached to its distal end to be placed in contact with the skin of the patient and the energy can be transferred to the tip from the energy source. The tip can be removable and does not or does not substantially interfere with the energy delivered from the probe. The probe can further comprise a sensor to monitor the position of the energy source relative to the spine in the patient. If the probe comprises a sensor, the device can further comprise a stereotactic system having an overhead camera for monitoring the sensor.

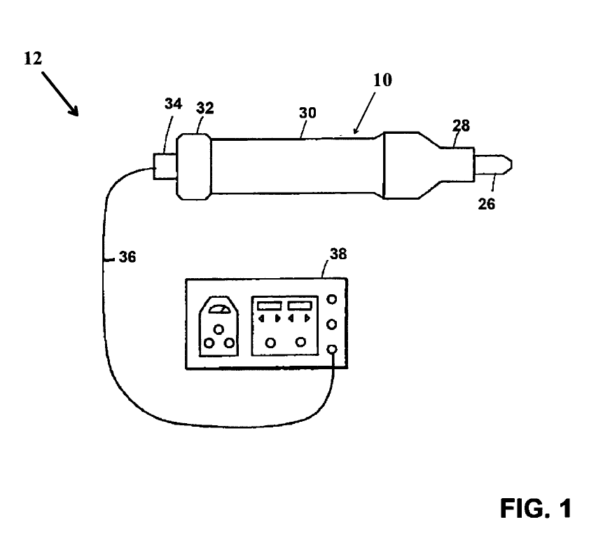

Referring to FIG. 1, one embodiment of a device 12 is provided that is configured to be moved along the skin of a patient near a pain generator or suspected pain generator and deliver energy through the skin of the patient to the target tissue site to produce a pain signal at or near the pain generator or suspected pain generator. The device 12 includes an external transducer probe 10 which is coupled to an energy source or generator 38 via a conduit or connecting wire 36. The probe 10 comprises a connector 34 which is an optional means for attaching the probe 10 to the conduit 36, a proximal end 32, a distal end 28, a body 30 between the proximal end 32 and the distal end 28, and a tip 26 which is placed in contact with the skin of a patient during use of the device. The proximal end 32, body 30 and distal end 28 make up the shape of a handle which makes it easier for the practitioner to manipulate the probe 10. During use of the device 12, the energy source 38 provides energy to the probe 10 via the conduit 36 and the energy is transmitted from the proximal end 32 of the probe 10 to its distal end 28 and out the tip 26. The tip 26 on the distal end 28 of the probe 10 is placed in contact with the skin in the direction of a pain generator or a suspected pain generator and the energy vibrates through the skin to the target tissue site with an intensity to produce a pain signal.

In some embodiments, the tip 26 is a quick attachment-detachment system. An ultrasonic probe device with a quick attachment-detachment system is described in U.S. Pat. No. 6,695,782 and co-pending patent applications U.S. Ser. No. 10/268,487 and U.S. Ser. No. 10/268,843, and the entirety of all these patents and patent applications are hereby incorporated herein by reference. In some embodiments, the tip 26 is attached by threading or reverse threading that can be turned to connect it. In some embodiments, the tip 26 can be a luer-lock connection.

The device allows the practitioner to properly diagnose the location of the pain generator as well as produce a pain map that locates the pain generator(s) so that the practitioner can rule out referred pain. For example, as spinal discs and facets begin to degenerate, blood vessels and nerve fibers grow into cracks of the annulus, facet capsules, or other anatomic structures. These nerve fibers cause pain during certain movements. Using a device such as this embodiment, the probe is positioned on the skin near or in the direction of a spinal disc or facet. The frequency of the probe is adjusted to vibrate the surrounding tissue and nerve fibers to induce a localized pain signal. In some embodiments, the energy such as ultrasound energy generated from the device will vibrate newly growing or sprouting nerve fibers and blood vessels that are growing in the body in an attempt to repair damaged tissue. The device and methods will cause vibration of these areas of new growth that are sensitive and a pain signal will be generated by the patient. The awake patient can notify the practitioner when a pain signal is produced (e.g., when pain is induced). The practitioner notes the location of the pain signal and then repositions the probe on the skin in the direction of another target tissue site at a pain generator or suspected generator. The frequency of the probe is adjusted to vibrate the surrounding tissue and nerve fibers to induce a localized pain signal. The awake patient can notify the practitioner when a pain signal is reproduced (e.g., when pain is induced for a second time). The practitioner notes the location of the pain signal and if the lower amount of the energy such as ultrasound energy was needed to produce a pain signal. If so, then the practitioner is approaching the pain generator.

Of course, the practitioner will minimize having to reposition the probe. The practitioner will, in some embodiments, first place the probe on the skin in the direction of the suspected pain generator(s). If a negative response or no pain signal is obtained, then the practitioner will move the probe on the skin in the direction of subsequent suspected areas of pain generator(s). The practitioner will use the results in a serial or process of elimination fashion. In this way, the practitioner can create a pain generator map and triangulate the one or more pain generators, which will aid in the diagnosis, prognosis and treatment of the spinal disease.

In some embodiments, if the spinal area is damaged and the probe is in contact with the skin near the damaged area, a lower amount of energy such as ultrasound energy is needed to vibrate the tissue near the damaged area and cause a pain signal. The practitioner would then know he or she is holding the probe in the direction of the pain generator. Thus, the first indicator of a pain source is a positive response from the patient, the amount of ultrasound would be an indictor of the severity of the pain source.

The medical device and methods described herein will be helpful for the practitioner to determine if the pain is being generated just from the facets or disc, and the practitioner can decide if a less invasive therapy such as conservative care, an epidural injection, drug depot implantation, posterior stabilization, or fusion is required. It will also provide information whether the pain is localized enough to result in a good clinical outcome or the pain is more diffuse requiring a multi-level spinal surgery.

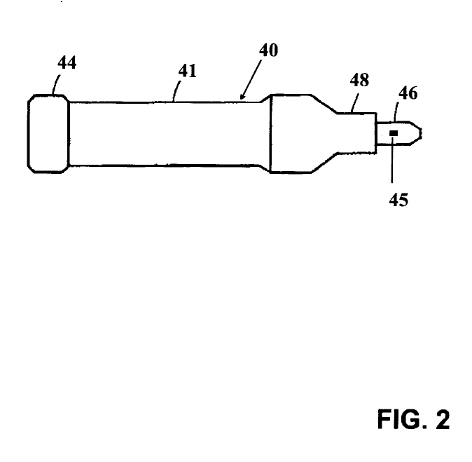

FIG. 2 shows an embodiment of an external transducer probe 40 having a distal end 48, a proximal end 44 and a body 41 therebetween. The probe 40 has the shape of handle wherein the practitioner can hold the probe 40 around the body 41. The body 41 of the probe 40 may have grips or wings (not shown) that make it easier for the practitioner to manipulate the probe 40. Those skilled in the art will understand that the probe 40 can have a variety of shapes that will enable a practitioner to manipulate it while use. The proximal end 44 of the probe engages an energy source (not shown) for receipt of energy for the probe 40. The distal end 48 is attached to a tip 46 which is placed in contact with the skin of a patient. In some embodiments, the tip 46 can be attached to the distal end 48 via a quick attachment-detachment system. The tip 46 is shown in FIG. 2 to have a sensor 45 which can be used to track the position and direction of the energy relative to the pain generator or suspected pain generator via an imaging device such as a CAT scan thereby aiding the practitioner in identifying the pain generator.

The tip 46 can be any shape including, but not limited to, rounded, bent, flat, pointed, smooth, blunt, ball, other shapes, or a combination thereof. The tip 46 may comprise an orifice or orifices that may deliver a coupling medium such as a liquid to the skin. The orifice or orifices may also be used to extract a coupling medium or fluid that is delivered to the skin. The orifice or orifices may be located anywhere on the tip 46. In some embodiments, the tip 46 can directly contact the skin and not use any coupling medium such as liquid. In some embodiments, the tip 46 of the probe 40 does not have an orifice. In some embodiments, the tip 46 may be held flat against the skin to diagnose the pain generator or suspected pain generator or it may be held at an angle. During application of energy to the skin, the probe tip 46 or any portion of the probe 40 may be moved latitudinally, moved longitudinally, rotated, lifted, or any combination thereof. If the tip 46 of the probe 40 is rotated during application, then energy may be delivered to the sides of the pain generator or suspected pain generator. If the tip 46 is lifted during application of the energy to the skin, then energy may be delivered from both the radial side of the tip 46 and the distal end of the tip 46. In some embodiments, the tip 46 is smooth to prevent damage to the skin.

Examples of tip styles include, for example, blunt tips, Trephine, Cournand, Veress, Huber, Seldinger, Chiba, Francine, Bias, Crawford, deflected tips, Hustead, Lancet, or Tuohey.

The tip 46 of the probe 40 has a sensor 45 as shown in FIG. 2. In some embodiments, the sensor 45 can be a LED sensor. In some embodiments, multiple LED sensors can be placed on or in the proximal end 44 or distal end 48 of the probe 40 or both. These LED sensors can be coupled to overhead cameras (not shown) so the overhead cameras can track the probe's position and direction and the position points from the probe's LED sensors can be superimposed onto a 3D CT reconstructed image of the spine on a computer. An example of a suitable system that the probe 40 can be coupled to or used in conjunction with is StealthStation® iNAV (available from Medtronic Sofamor Danek, Memphis, Tenn.) which allows tracking of the energy. In this way, the practitioner will know exactly where he/she is at and the source of pain, when a positive pain response occurs, thus identifying the position of the pain generator. In some embodiments, two or more probes can be used and each can include a sensor such as a LED sensor. The use of two or more probes allows the practitioner to triangulate the location of the pain generator by setting up an increased integrated signal at a location within the body. This allows for an accurate determination of the location of the pain generator via steriotactic methods for tracking of the sensor and probe's location and direction.

### Energy

The probe vibrates the tissue surrounding the pain generator to induce one or more pain signals. The energy source can be ultrasound energy or another energy capable of transmitting a mechanical wave. If the energy is ultrasound energy, the ultrasound energy is directed to the tissues, vibrates the tissues and does not destroy, ablate or bore holes or bore cavities through the tissues. The dose of the ultrasound energy can be set to be high and low frequencies. In some embodiments, the ultrasound is applied to the skin of a patient for 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 55 or 60 seconds or a time sufficient to generate a pain signal. Of course, the more damage to the tissue the quicker the time for a pain response. If there is no pain response within 10 seconds, then the practitioner knows he or she is not manipulating the probe on the skin at a point in the direction of a pain generator.

The intensity of the ultrasound energy can be controlled through a variation in the ultrasound parameters such as the frequency, the amplitude, and the treatment time. The frequency range for the ultrasound energy can be, for example, 15 kHz to 20 MHz. In some embodiments, the low-frequency ultrasound range is 20 kHz-100 kHz, 25 kHz-50 kHz, or 30 kHz-50 kHz. In some embodiments, the ultrasound energy emitted by the probe can be a high-frequency ultrasound energy in the range of about 0.7 MHz-3 MHz, 0.5 MHz-1.0 MHz, 0.7 MHz-1.0 MHz, 1.0 MHz-10.0 MHz, or 5.0 MHz-10.0 MHz. The amplitude of the low-frequency ultrasound energy can be 5 microns and above, with the preferred low-frequency amplitude to be in range of 30 microns to 100 microns, and the recommended low-frequency amplitude value is 100 microns. The amplitude of the high-frequency ultrasound energy can be 1 micron and above, with the preferred high-frequency amplitude to be at least 5 microns, and the recommended high-frequency amplitude value is 10 microns. Thus, the probe can be set to emit the above doses of high and low frequency ultrasound energy. There are a variety of factors that may influence the dosage given to the target skin site. These factors may include the type of tissue being treated, the condition of the spine, and the size of a spinal defect or location.

In some embodiments, the probe can be set depending on the type of tissue near the pain generator or suspected pain generator. For example, the probe can be set to deliver high or low frequency ultrasound depending on the type of tissue the pain generator or suspected pain generator is at or near (e.g., annulus, nucleus, facet capsule, nerve root, etc.).

In some embodiments, a method is provided where the probe is re-positioned on the skin in the direction of a suspected pain generator a plurality of times before it is positioned to provide energy directly to or near the pain generator so that the location of the pain generator is diagnosed. Of course, the practitioner will try to limit the number of times the probe is repositioned. In some embodiments, a method is provided where the probe is re-positioned on the skin in the direction of a plurality of suspected pain generators before it is re-positioned on the skin at a position in the direction of a plurality of pain generators so that the location of the pain generators can be mapped.

In some embodiments, the method involves targeting a target tissue site to determine if one or more pain generators are present. Ultrasonic energy is provided at a first dose to the target tissue site by placing the probe on the skin at a point in the direction of the target tissue site. If the first dose is sufficient to stimulate pain, the practitioner then notes the location and verifies that the stimulated target tissue is a pain generator and then moves to another location of the pain generator or suspected pain generator. If there is no pain signal (e.g., there is no pain or the scaled pain score is lower), the practitioner can increase the ultrasonic dosage until a pain signal is received. If no pain signal is exhibited by the patient, the practitioner can note that no pain generator was diagnosed and reposition the probe on the skin in the direction of another target tissue area. The dose that is applied to the target tissue site can be gradually increased, decreased or remain the same to diagnose the pain generator. Based on the patient's response, for example, if the ultrasound dose is lower and the patient experiences more pain when the probe is placed on the skin nearing the direction of the target tissue site, the practitioner notes this and now knows that the position of the probe on the skin is getting closer to being directly pointing to the pain generator or the probe is pointed directly at the pain generator. The probe can be repositioned and the second target tissue area tested with the same or decreased dose and if the pain signal from the patient is greater (e.g., the scaled pain score is higher), the practitioner notes this and now knows that the position of the probe on the skin is getting closer to being directly pointing to the pain generator or the probe is pointed directly at the pain generator. Likewise, the probe can be repositioned and the third target tissue area tested with the same or increased dose and if the pain signal from the patient is less, then the practitioner notes this and now knows that the position of the probe on the skin is getting farther from being directly pointed to the pain generator. This procedure can be repeated a plurality of times. In this way, many focal points of the pain generator and non-pain generator can be mapped and true pain generators can be mapped and diagnosed.

In some embodiments, by using the devices and methods described herein, the practitioner can rule out referred pain—that is where the pain sensation is localized to an area completely unrelated to the site of injury. Thus, the location of the pain generator is undetermined as no definitive diagnosis can be made and the practitioner will now monitor the patient and examine other areas to determine the location of the pain generator. It will be understood by one of ordinary skill in the art that the pain the patient experiences can be assessed objectively and subjectively, using pain scores and other methods well-known in the art.

If the pain generator cannot be isolated, the practitioner will perform further physical examinations to determine the location of the pain generator. In some embodiments, the practitioner will look for irritation, injury, inflammation, limited mobility or range of motion due to pain, abnormalities in the strength and sensation of particular parts of the body, further neurologic and radiologic examinations (e.g., MRI, CT, CT myelography, etc.) to explain the persistent pain symptoms the patient experiences. Such pain can exhibit as, for example, radiating pain, weakness, or numbness in the legs, back, arms, or neck. This will allow the practitioner to identify additional suspected pain generators. In this way, the practitioner can use the devices and methods to selectively narrow in on the pain generator (e.g., pain generators in nerve areas, facet joints, medial branch nerves, sacroiliac joints, discs) and rule out selective areas where the pain generator is not located. When the patient experiences pain at the site where the ultrasound is directed to through the skin and the pain generator identified, this will allow the practitioner to diagnose the location of the pain generator. A surgical procedure can now be scheduled to provide long-term relief at the site of the pain generator. In this way, operating on the wrong area and/or suspected pain generator is avoided.

In some embodiments, the patient can be monitored for pain. This can be accomplished by the patient themselves or by the practitioner. As or after the energy is applied, the pain symptoms can be monitored for severity or intensity of pain, reduced disability or increased mobility, alleviation of one or more symptoms associated with the pain, reduced resting pain, shortened duration of pain symptoms, reduced pain sensitivity or sensation in a given time frame. This can alert the practitioner that he/she has successfully found a pain generator.

In some embodiments, the devices and methods described herein are utilized to detect pain generators for vertebral abnormalities, such as, compression fractures, pars defects, vertebral instability, soft tissue abnormalities in ligaments, tendons, annulus, muscles, cartilaginous structures, joints (e.g., facet joints, intervertebral discs, sacroiliac joints, etc.) or abnormalities resulting from tumors, infection or other infiltrative processes. Pain generators resulting from nerve root lesions (e.g., compressive lesions from adjacent discs, hypertrophic facet joints, facet joints cysts, faulty hardware positioning, bony foraminal encroachment, spondylolisthesis, spondylolysis, congenitally short pedicles, nerve sheath tumors, granulation tissue and/or arachnoiditis, etc.), spinal nerve compression (e.g., spinal stenosis), peripheral nerve lesions, femoral neuropathy, meralgia paresthetica, peroneal neuropathy, asymmetrical neuropathies, lower limb joint pathology, vascular pathology, degenerative disc and joint disease or the like. In some embodiments, the methods and devices herein can be used to diagnose the cause of back pain.

In some embodiments, the energy such as ultrasound energy may be applied to the skin in the direction of the pain generator or suspected pain generator at a first dosage level sufficient to stimulate the pain generator or suspected pain generator by vibrating the tissue. The ultrasonic energy can be applied as a continuous wave of ultrasound energy from the probe, starting at a zero or quiescent amplitude, and slowly increasing the amplitude until a pain response is noted. Alternatively, the ultrasound energy can be applied in pulse dosing. The pain signal can be verified, for example, by having the patient in a conscious or semi-conscious state and asking the patient when the stimulation from the ultrasonic energy is felt, and asking the patient if the stimulation is at the same location as the pain. In another embodiment, a measurement of neural activity corresponding to the pain may be performed, or a measurement of an electrophysiological signal associated with the pain may be performed to measure the pain signal.

In some embodiments, after the pain generators are isolated and the locations are mapped, treatments of the area can be performed. In this way, the location of the pain generator can be confirmed and the practitioner will know that he/she is operating at the right location. For example, the practitioner now knows the pain generator is between lumbar vertebrae L3 (lumbar 3) and L4 (lumbar 4), and can operate on these levels instead of the wrong level (e.g., L1 and L2).

In some embodiments, the methods and devices described herein can be used with implantable drug depots. For example, a drug depot can be implanted at or near the pain generator and can release a pain reliever or anti-inflammatory agent over a period of about 2 weeks to about 4 weeks or from about 3 days to 4 weeks to reduce the pain.

In some embodiments, a method is provided for diagnosing a pain generator at or near the spine in a patient suffering from pain, the method comprising: positioning a probe on the skin in the direction of a target tissue site, the probe having a proximal end, a distal end and a body therebetween, the probe supplied with ultrasonic energy from an ultrasonic energy source via a conduit; delivering a first dose of ultrasonic energy from the distal end of the probe on the skin of a patient in the direction of the target tissue site at or near the spine to produce a first pain signal; re-positioning the probe on the skin in the direction of a second target tissue site at or near the spine; delivering a second dose of ultrasonic energy from the distal end of the probe on the skin in the direction of the second target tissue site at or near the spine to produce a second pain signal; and comparing the first dose and the first pain signal to the second dose and the second pain signal to diagnose the pain generator or suspected pain generator, wherein a greater pain signal indicates the presence of the pain generator.

### Back Pain

Back pain is a very difficult pain for practitioners throughout the world to treat. The reason often is inadequate diagnosis, or failure to identify the true pain generator. By using a device and method provided herein, proper diagnosis of the location of the pain generator can be made. Thus, the practitioner can identify the pain generator causing the back pain. For example, lumbar spine pain falls into two general categories: purely axial, and radicular or radiating. In patients with purely midline or lateralizing low-back pain, common and uncommon etiologies should be considered. These include pain originating from osseous structures (vertebral compression fractures, pars defects, or vertebral instability), soft tissue (ligaments, tendons, muscles and cartilaginous structures), joints (facet joints, intervertebral discs, sacroiliac joints) as well as tumors, infection or other infiltrative processes. In patients with radicular pain, nerve root lesions (compressive lesions from adjacent discs, hypertrophic facet joints, facet joints cysts, faulty hardware positioning, bony foraminal encroachment caused by spondylolisthesis or spondylolysis, congenitally short pedicles, nerve sheath tumors, granulation tissue and arachnoiditis), spinal nerve compression and peripheral nerve lesions should be considered. By mapping the pain generators and non-pain generators using the devices and methods provided herein, a more definitive diagnosis can be made as to the cause of the pain in the above conditions.

In some embodiments, the devices and methods can be used to confirm diagnosis that does not originate from the spine but is often misdiagnosed (often as classified as referred pain) such as, for example, femoral neuropathy, meralgia paresthetica, peroneal neuropathy, asymmetrical neuropathies, lower limb joint pathology or vascular pathology. These conditions are often mistaken for radicular pain in patients with and without radiographic evidence of degenerative disc and joint disease. Using a diagnostic device and method of the present application, these conditions now can be more definitively diagnosed.

In some embodiments, the back pain can be caused by a herniated disc. Typically, when a patient has a herniated disc, the patient will exhibit severe or persistent radicular pain. When the herniated disc is in the lower back, persistent pain can originate in the back and often extends (“radiates”) into the leg along the distribution of the sciatic nerve (lumbar radicular pain, or sciatica). In patients with a herniated disc in the neck, the persistent pain can originate in the neck and often radiates into the arm. Patients can be diagnosed with herniated disc through a variety of characteristic findings. These include, for example: persistent radiating pain; characteristic findings on a physical exam indicative of nerve root irritation, injury or inflammation, such as limited mobility or range of motion due to pain; abnormalities in the strength and sensation of particular parts of the body that are found with a neurological examination, radiologic examination suggestive of a herniated disc. The devices and methods of the present application can assist the practitioner in confirming the location of the pain generator—in this case the herniated disc. Subsequently, a surgery can be performed and partial removal of the nucleus pulposus or annulus fibrosus can be performed. The devices and methods will guide the practitioner and he/she will now know that the right location is being operated on.

Spinal stenosis is another condition where the patient will exhibit, among other things, back pain. Spinal stenosis, either acquired or congenital, results from degenerative changes in the spine, variably including the intervertebral disks, the intervertebral joints (facet joints) and the ligamentum flavum. In each case, the degenerative changes together result in a gradual narrowing of the lumbar or cervical spinal canal, causing compression of the spinal cord and spinal nerve roots. Symptoms include: pain and/or numbness in the neck, back, buttocks, legs, thighs or calves that is worse with walking, standing and/or exercise; back pain that radiates to the legs; weakness of the legs; and difficulty or imbalance when walking. Patients can be diagnosed with spinal stenosis through, for example, persistent radiating pain; neurologic examination findings of abnormal sensation and muscle weakness in the legs; gait disturbances and characteristic bent over posture; asymmetric deep tendon reflexes; and radiologic findings of spinal stenosis by x-ray (e.g., myelogram), MRI, spinal CT or CT myelography or the like. Depending on whether the stenosis is central or foraminal, provocative maneuvers on physical examination such as side bending reproducing the pain may be negative or positive, respectively. In some embodiments, the devices and methods may be used to diagnose the pain generators for the facet degeneration.

The devices and methods of the present application can assist the practitioner in confirming the location of the pain generator—in this case the abnormality causing the spinal stenosis. Subsequently, a surgery can be performed to treat the spinal stenosis. The practitioner will now know that the right location is being operated on and long term pain relief can be provided to the patient.

In some embodiments, the devices and methods of the present application can be used to triangulate the pain generators. Thus, a plurality of probes (at least two, at least three, at least four, at least five, at least six, at least seven, etc.) can be placed on the skin in the direction of the target tissue site (also known as the pain generator or pain generation site) such that the target tissue site falls within a region that is either between the probes when there are two, or within an area whose perimeter is defined by a set of plurality of probes.

### Kits

In various embodiments, a kit is packaged housing an external transducer probe, one or more conduits, an energy source, etc. The kit may include additional parts along with the components of a device, for example, disposable probe tips, cannulas and/or drug depots combined together to be used to detect one or more pain generators as well as treat the pain generators. The kit may include the probe in one compartment and the energy device and a conduit in a second compartment. The third compartment may include drug depots or each drug depot with a different release profile may be labeled and placed in a different compartment (e.g., bolus dose depot compartment, sustained release depot compartment, etc.) and any other instruments needed for the localized drug delivery. A third compartment may include gloves, drapes, wound dressings and other procedural supplies for maintaining sterility, as well as an instruction booklet and a DVD showing how the device operates. A fourth compartment may include additional cannulas and/or needles for administration of the drug depots. Each tool may be separately packaged in a plastic pouch that is radiation sterilized. A fifth compartment may include disposable probe tips for the probe and the probe tips may include a sensor. A cover of the kit may include illustrations of the probe procedure and a clear plastic cover may be placed over the compartments to maintain sterility.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A device for diagnosing a pain generator or a suspected pain generator at or near the spine in a patient suffering from pain, the device comprising an external transducer probe having a proximal end, a distal end and a body therebetween, the probe configured to be placed in contact with the skin of the patient near the pain generator or suspected pain generator; an energy source that supplies the probe with energy; and a conduit for engaging the energy source and the proximal end of the probe; wherein the energy is transferred from the energy source to the distal end of the probe and has an intensity to deliver and produce a pain signal when at least the distal end of the probe is pointing in the direction of the pain generator or suspected pain generator.

2. A device according to claim 1, wherein the energy is ultrasound energy and the ultrasound energy has a frequency in the approximate range of 15 kHz-20 MHz, 20 kHz-100 kHz, 25 kHz-50 kHz, 30 kHz-50 kHz, 1 MHz-10 MHz, 5 MHz-10 MHz or 500 kHz-1 MHz.

3. A device according to claim 1, wherein the probe further comprises a tip attached to the distal end to be placed in contact with the skin of the patient and the energy is transferred to the tip from the energy source.

4. A device according to claim 3, wherein the tip is removable.

5. A device according to claim 1, wherein the probe comprises a sensor to monitor the position of the energy source relative to the spine in the patient.

6. A device according to claim 6, further comprising a stereotactic system having an overhead camera for monitoring the sensor.

7. A device according to claim 1, wherein (i) the probe is re-positioned near the suspected pain generator a plurality of times before it is positioned on the skin of the patient at a position in the direction of the pain generator, so that a location of the pain generator is diagnosed or (ii) the probe is re-positioned at a plurality of suspected pain generators before it is re-positioned on the skin of the patient at a position in the direction of a plurality of pain generators so that locations of the pain generators can be mapped.

8. A device according to claim 1, wherein the distal end of the probe contacts the skin of the patient and thereby vibrates a tissue having the pain generator or the suspected pain generator at or near the spine to generate a pain signal.

9. A device according to claim 8, wherein the tissue is at least one muscle, ligament, tendon, cartilage near the spine, or spinal nerve, spinal disc, facet joint, spinal foraminal space near the spinal nerve root, facet, annulus, or spinal canal.

10. A method for diagnosing a location of a pain generator or a suspected pain generator at or near the spine in a patient suffering from pain, the method comprising:
positioning an external transducer probe on the skin of the patient in the direction of the pain generator or suspected pain generator, the probe having a proximal end, a distal end and a body therebetween, the probe supplied with energy from an energy source via a conduit that connects the energy source to the proximal end of the probe; and
delivering sufficient energy from the distal end of the probe through the skin of the patient to a tissue at or near the pain generator or suspected pain generator to produce a pain signal so as to diagnose the location of the pain generator or suspected pain generator.

11. A method according to claim 10, wherein the probe is re-positioned at a suspected pain generator a plurality of times before it is positioned on the skin of the patient at a position in the direction of the pain generator so that the location of the pain generator is diagnosed.

12. A method according to claim 10, wherein the energy is ultrasound energy and the ultrasound energy has a frequency in the approximate range of 15 kHz-20 MHz, 20 kHz-100 kHz, 25 kHz-50 kHz, 30 kHz-50 kHz, 1 MHz-10 MHz, 5 MHz-10 MHz or 500 kHz-1 MHz.

13. A method according to claim 10, wherein the probe further comprises a tip attached to the distal end to be placed in contact with skin of the patient and the energy is transferred to the tip from the energy source.

14. A method according to claim 10, wherein the probe comprises a sensor to monitor the position of the energy source relative to the spine in the patient.

15. A method according to claim 14, further comprising administering a stereotactic system having an overhead camera for tracking the sensor.

16. A device for diagnosing a pain generator or a suspected pain generator at or near the spine in a patient suffering from pain, the device comprising:
(a) a first external transducer probe having a proximal end, a distal end and a body therebetween, the first probe configured to be placed in contact with the skin of the patient at a first location in the direction of the pain generator or suspected pain generator;
(b) a second external transducer probe having a proximal end, a distal end and a body therebetween, the second probe configured to be placed in contact with the skin of the patient at a second location in the direction of the pain generator or suspected pain generator;
(c) an energy source that supplies the first probe and the second probe with energy;
(d) a first conduit for engaging the energy source and the proximal end of the first probe; and
(e) a second conduit for engaging the energy source and the proximal end of the second probe;
wherein the energy is transferred from the energy source to the distal end of the first probe and the distal end of the second probe, the energy from both the first probe and the second probe is of an intensity for each probe to deliver a signal through the skin of the patient to or toward the pain generator or the suspected pain generator when the distal end of each probe is pointing in the direction of the pain generator or the suspected pain generator, and the signals produced by each probe intersect at the location of the pain generator or the suspected pain generator.

17. A device according to claim 16, wherein the energy is ultrasound energy and the ultrasound energy has a frequency in the approximate range of 15 kHz-20 MHz, 20 kHz-100 kHz, 25 kHz-50 kHz, 30 kHz-50 kHz, 1 MHz-10 MHz, 5 MHz-10 MHz or 500 kHz-1 MHz.

18. A device according to claim 16, wherein the first probe further comprises a removable tip attached to its distal end to be placed in contact with the skin of the patient, the second probe further comprises a removable tip attached to its distal end to be placed in contact with the skin of the patient, and the energy is transferred to the tip of the first probe from the energy source and the tip of the second probe from the energy source.

19. A device according to claim 16, wherein each probe comprises a sensor to monitor the position of the energy source relative to the spine in the patient.

20. A device according to claim 19, further comprising a stereotactic system having an overhead camera for monitoring the sensors.
